(19) United States
(12) Patent Application Publication
(10) Pub. No.: US 2020/0237570 A1
(43) Pub. Date: Jul. 30, 2020

(54) BIODEGRADABLE UNDERWEAR

(71) Applicant: LIATI D.I.A.N LTD., Yahud (IL)

(72) Inventor: Liat ZIMERING, Yehud (IL)

(73) Assignee: LIATI D.I.A.N LTD., Yahud (IL)

(21) Appl. No.: 16/652,987

(22) PCT Filed: Sep. 3, 2018
(86) PCT No.: PCT/IL2018/050979

§ 371 Date: Apr. 2, 2020

Related U.S. Application Data
(60) Provisional application No. 62/566,548, filed on Oct. 2, 2017.
(60) Provisional application No. 62/624,828, filed on Feb. 1, 2018.
(60) Provisional application No. 62/668,823, filed on May 9, 2018.

(51) Int. Cl. A61F 13/15; A61F 13/49; A61F 13/505; A61F 13/64

(52) U.S. Cl. CPC A61F 13/15252; A61F 13/49003; A61F 2013/424; A61F 13/64; A61F 13/505

(57) ABSTRACT

Disclosed is biodegradable underwear having side fastening and unfastening means, wherein the biodegradable underwear is possibly coupled with a biodegradable sanitary napkin, a sensor or both. Disclosed also is a biodegradable sanitary napkin comprising a sensor. Further disclosed is biodegradable underwear comprising a belt and a biodegradable garment.

### FIELD OF THE INVENTION

The present invention is directed to biodegradable underwear with side fastening/un-fastening means, such as clasps, ties, hooks, fabric hook and loop fasteners, tape, snaps, buttons, zippers and the like. The invention is further directed to biodegradable underwear with side fastening/unfastening means further comprising a biodegradable sanitary napkin, a sensor or both. In addition, the invention is directed to a biodegradable sanitary napkin incorporating a sensor. The invention is further directed to biodegradable underwear comprising a belt and a biodegradable garment.

### BACKGROUND OF THE INVENTION

The use of biodegradable materials had increased over the past years due to the environmentally beneficial properties of such materials. Such materials are now commonly used in the manufacture of a wide range of products, including various types of plastic bags and other forms of packaging.

Recently, biodegradable fabrics have been designed as well. Such biodegradable fabrics may be prepared from bamboo fibers, cotton, or any other appropriate natural materials.

Further, biodegradable sanity napkins have also been developed lately, increasing the possibility of the use of environmentally friendly products.

Nonetheless, although developed, biodegradable sanitary napkins are not widely used and certainly not in combination with biodegradable fabrics. Further, the removal of underwear generally requires removing additional garments, such as pants, tights and the like. The removal of shoes is necessary in many instances as well. As is well known, the removal of additional apparel in order to remove/apply underwear proves to be cumbersome, especially for younger and older parties or for parties in uncomfortable situations, such as soldiers in a battle field and the like, as well as for physically impaired people.

Therefore, it would be highly desirable to provide biodegradable underwear that does not require the removal of additional garments and that may further be combined with biodegradable sanitary napkins and/or sensors, such as humidity sensors.

### SUMMARY OF THE INVENTION

Embodiments of the invention are directed to a biodegradable underwear comprising side fastening/unfastening means.

According to some embodiments, the biodegradable underwear further comprises a biodegradable sanitary napkin. According to some embodiments, the biodegradable underwear further comprises a sensor. According to some embodiments, the biodegradable underwear further comprises a biodegradable sanitary napkin.

According to some embodiments, the sensor is coupled to a designated application. According to some embodiments, the designated application is installed on a smartphone. According to some embodiments, the designated application alerts a user when the biodegradable underwear and/or the biodegradable sanitary napkin should be replaced.

According to some embodiments, the sensor is a humidity sensor. According to some embodiments, the biodegradable sanitary napkin is sewn into or onto the biodegradable underwear.

Further embodiments of the invention are directed to a biodegradable sanitary napkin comprising a sensor. According to some embodiments, the sensor is coupled to a designated application. According to some embodiments, the designated application is installed on a smartphone. According to some embodiments, the designated application alerts a user when the biodegradable sanitary napkin should be replaced. According to some embodiments, the sensor is a humidity sensor.

Additional embodiments of the invention are directed to an application for communication with a user and a sensor, wherein the sensor is installed in or on biodegradable underwear or a biodegradable sanitary napkin, and wherein the application is enabled to:
receive data from the sensor;
interpret the data;
inform the user of the interpretation;
be updated by the user;
provide data from the sensor, interpretation of such data, or both, upon receiving queries from the user;
alert the user of necessary actions; and
remind the user of necessary actions.

Further embodiments of the invention are directed to a system comprising:
biodegradable underwear;
a biodegradable sanitary napkin;
a sensor; and
an application for communication with a user and the sensor, wherein the sensor is installed in or on the biodegradable underwear or the biodegradable sanitary napkin, and wherein the application is enabled to:
receive data from the sensor;
interpret the data;
inform the user of the interpretation;
be updated by the user;
provide data from the sensor, interpretation of such data, or both, upon receiving queries from the user;
alert the user of necessary actions; and
remind the user of necessary actions.

Further embodiments of the invention are directed to a kit comprising:
a belt;
a biodegradable garment; and
fastening/unfastening means to attach the biodegradable garment to the belt.

According to some embodiments, the kit further comprises a sanitary napkin. According to some embodiments, the kit further comprises a sensor. According to some embodiments, the fastening/unfastening means are selected from the group comprising: clasps, ties, hooks, fabric hook and loop fasteners, tape, snaps, buttons, zippers or any combination thereof.

Further embodiments of the invention are directed to biodegradable underwear comprising a belt, a biodegradable garment and fastening/unfastening means for attaching/detaching the biodegradable garment to/from the belt. According to some embodiments, the belt is reusable. According to some embodiments, the biodegradable underwear comprises a sensor, wherein the sensor is prepared from conductive ink printed onto the biodegradable underwear in a pattern. According to some embodiments, the belt is disposable. According to some embodiments, the belt, the biodegradable underwear, the fastening/unfastening means for attaching/detaching the biodegradable garment to/from the belt, or any combination thereof, are designed as one piece.

According to some embodiments, the biodegradable underwear and/or the biodegradable garment is prepared from seaweed, zinc, a biodegradable polymer, any other type of biodegradable compounds, or any combination thereof. According to some embodiments, the biodegradable sanitary napkin is prepared from seaweed, zinc, a biodegradable polymer, any other type of biodegradable compounds, or any combination thereof.

### BRIEF DESCRIPTION OF THE DRAWINGS

The above and other characteristics and advantages of the invention will be better understood through the following illustrative and non-limitative detailed description of preferred embodiments thereof, with reference to the appended drawings, wherein:

FIG. 1 presents an embodiment of the biodegradable underwear of the invention comprising fastening/unfastening means;

FIG. 2 presents an embodiment of the biodegradable underwear of the invention comprising fastening/unfastening means as well as a biodegradable sanitary napkin incorporated into the biodegradable underwear;

FIG. 3 presents an embodiment of the biodegradable underwear of the invention comprising fastening/unfastening means as well as a sensor and a biodegradable sanitary napkin incorporated into the biodegradable underwear;

FIG. 4 presents an embodiment of the invention including a biodegradable sanitary napkin incorporating a sensor;

FIG. 5 presents a flow chart depicting an embodiment of the invention including the correspondence between the application and the sensor;

FIG. 6 presents an embodiment of the invention including a belt and a biodegradable garment in the detached configuration;

FIG. 7 presents an embodiment of the invention including a belt and a biodegradable garment in the attached configuration;

FIG. 8 presents the front view of an embodiment of the biodegradable underwear;

FIG. 9 presents the back view of an embodiment of the biodegradable underwear;

FIG. 10 presents the top view of an embodiment of the belt; and

FIG. 11 presents the top view of an embodiment of the biodegradable garment.

### DETAILED DESCRIPTION OF THE INVENTION

Unless otherwise defined, all technical and scientific terms used herein have the same meaning as commonly understood by one of ordinary skill in the art to which the invention pertains. In case of conflict, the specification, including definitions, takes precedence.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

It is to be noted that, as used herein, the singular forms “a”, “an” and “the” include plural forms unless the content clearly dictates otherwise. Where aspects or embodiments are described in terms of Markush groups or other grouping of alternatives, those skilled in the art will recognize that the invention is also thereby described in terms of any individual member or subgroup of members of the group.

As used herein, when a numerical value is preceded by the term “about”, the term “about” is intended to indicate +1-10%.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

The term “biodegradable” as used herein is to be understood to include any article that degrades through the action of living organisms, air, water or any combinations thereof within 60, 120 or 180 days in a controlled-condition industrial compost facility with high ventilation and controlled humidity.

The terms “underwear” and “biodegradable underwear”, as well as the terms “sanitary napkin” and “biodegradable sanitary napkin” are respectively interchangeable herein, unless specifically mentioned otherwise or unless would be understood to have a different meaning by a person skilled in the art. According to some embodiments, the biodegradable underwear and biodegradable sanitary napkin may be used by women during menstruation. According to some embodiments, the biodegradable sanitary napkin absorbs fluids, e.g., menstrual blood or urine, for up to 2, 4, 6, 8, 10, or 12 hours, depending on the volume of liquid released over time.

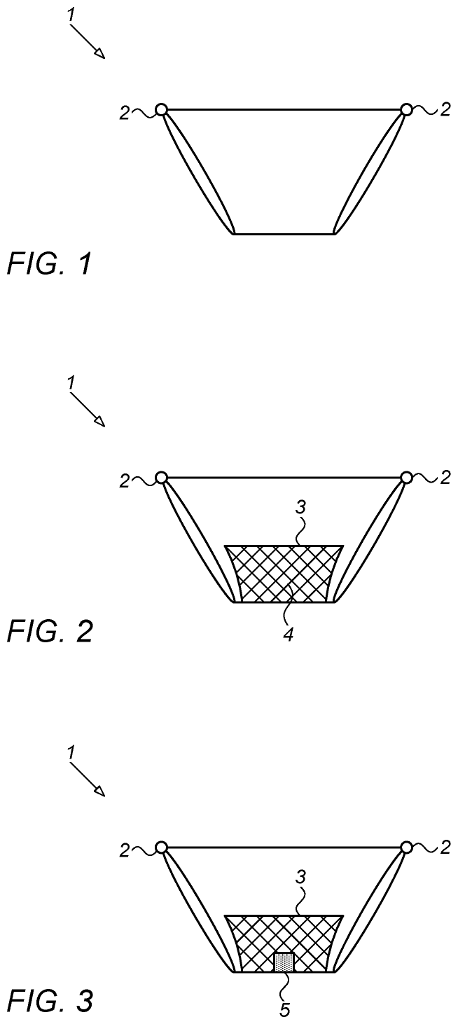

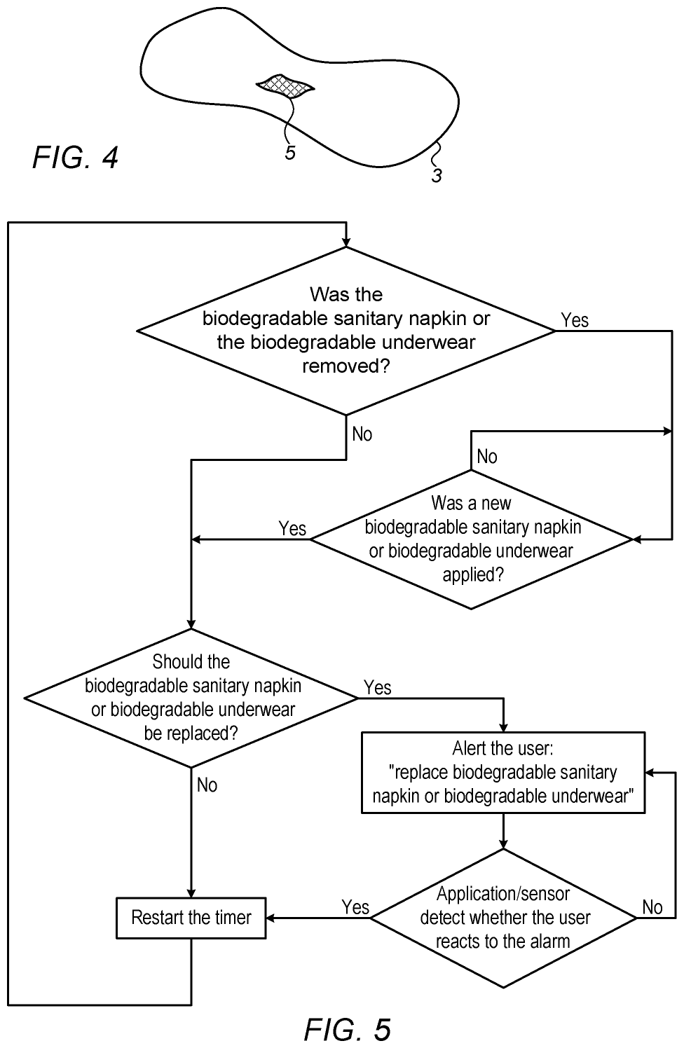

Reference is now make to FIG. 1, presenting an embodiment of the biodegradable underwear (1) comprising side fastening/unfastening means (2), wherein, as shown in FIG. 1, side fastening/unfastening means (2) are positioned on both sides of the biodegradable underwear, such that the underwear may be removed without removing any additional garments or shoes.

According to some embodiments, the side fastening/unfastening means are selected from clasps, ties, hooks, fabric hook and loop fasteners, tape, snaps, buttons, zippers and the like. According to some embodiments, each one of the side fastening/unfastening means may be designed differently, e.g., one side may include clasps, while the other side includes a tie, or any other possible combination.

According to further embodiments, the biodegradable underwear is designed such that the side fastening/unfastening means are not only functional, but also add to the esthetic design of the underwear, e.g., the ties may be tied in bows, possible in a contrasting color to the underwear itself.

According to some embodiments, the biodegradable underwear have a fastened form and an unfastened form, wherein they are transformed from one form to the other by fastening/unfastening the fastening means. The biodegradable underwear may be applied in the unfastened form, wherein after application and proper positioning on the body of the user, they are fastened. When removal of the biodegradable underwear is desired, they may be unfastened and subsequently removed. Therefore, it is possible to both apply and remove the biodegradable underwear in the unfastened form, such that the removal of other garments and/or shoes is not required. Further, bending down and/or raising the feet, as necessary with regular underwear, is also not required due to the fastening/unfastening means. According to some embodiments, the biodegradable underwear is applied in the fastened form and removed in the unfastened form, or vice versa, as desired by the user.

Such biodegradable underwear, including fastening/unfastening means may conceivable be used by any type of user, including young children who find the use of underwear cumbersome, older people, who may not be agile enough for the use of regular underwear, soldiers in the battle field and children in boarding schools, who don't always have the privacy to change cloths, physically impaired people, including patients after surgery that limits their movements, etc. it may also be conceivably used by young teenage girls, or older girls or women during menstruation.

According to some embodiments, the biodegradable underwear may remain on the user until a time at which the user desires to remove the biodegradable underwear. The biodegradable underwear may be removed and replaced with a different pair of underwear. According to other embodiments, the biodegradable underwear may be removed, or partially removed, in order to allow the users to relieve themselves, after which the biodegradable underwear may be returned to its original position on the body of the user.

As detailed herein, according to some embodiments, the biodegradable sanitary napkin is designed as an element of the biodegradable underwear, and is not removed separately. For example the biodegradable sanitary napkin may be irreversibly coupled to the biodegradable underwear by glue, lamination, stitching and the like. According to some embodiments, the biodegradable underwear is prepared from biodegradable fabric. According to some embodiments, the biodegradable fabric comprises seaweed, zinc, biodegradable polymers, or any other type of biodegradable compounds and any combination thereof.

As presented in FIG. 2, a biodegradable sanitary napkin (3) may be incorporated into biodegradable underwear (1). Biodegradable sanitary napkin may be incorporated into biodegradable underwear by any appropriate means, including adhesives, sewing, fabric hook and loop fasteners, snaps, tape, and the like. According to some embodiments, biodegradable sanitary napkin (3) is incorporated into biodegradable underwear (1) by sewing, e.g., using crisscross sewing pattern (4).

According to some embodiments, biodegradable sanitary napkin (3) is placed between two layers of the biodegradable fabric from which biodegradable underwear (1) is prepared such that the human body does not come in contact with biodegradable sanitary napkin (3). According to some embodiments, biodegradable sanitary napkin (3) is placed in a pocket formed from two layers of the biodegradable fabric, wherein, once placed in the pocket, biodegradable sanitary napkin (3) remains therein, unless physically removed therefrom.

According to some embodiments, biodegradable sanitary napkin (3) may be removed from biodegradable underwear (1) and possibly replaced by a new biodegradable sanitary napkin (3), without removing biodegradable underwear (1) from the body. According to some embodiments, the pocket includes any type of appropriate fastening means intended to ensure that biodegradable sanitary napkin (3) remains within the pocket, e.g., fabric hook and loop fasteners at the opening of the pocket, or the like. According to some embodiments, biodegradable sanitary napkin (3) is sewn into the pocket, possibly using crisscross sewing pattern (4). The addition of a biodegradable sanitary napkin may prove beneficial for teenagers and women during menstruation, patients suffering from any degree of incontinence and the like.

Reference is now made to FIG. 3 in which an embodiment including sensor (5) is presented. Particularly, according to some embodiments, biodegradable underwear (1) includes both biodegradable sanitary napkin (3) and sensor (5). Sensor (5) may be a sensor for detecting humidity or any necessary fluids, such as urine, blood or water. According to some embodiments, sensor (5) detects color, pH, odor and/or any other variables that may change when the biodegradable sanitary napkin (3) is soiled. According to some embodiments, sensor (5) is placed under sanitary napkin (3) and/or under a layer of the biodegradable fabric from which biodegradable underwear is prepared, such that the human body does not come in contact with sensor (5). Sensor (5) may be incorporated into biodegradable underwear (1) by any appropriate means, such as adhesives, sewing, fabric hook and loop fasteners, and the like. According to some embodiments, sensor (5) is prepared, at least partially, from a flexible material, such that it is comfortably worn and generally not noticed by the user.

According to some embodiments, sensor (5) transmits data to a dedicated application, e.g., installed on a smartphone. According to some embodiments, the application contacts the sensor at predetermined intervals, such as every half an hour, every hour, every two hours and the like. According to some embodiments, sensor (5) is a humidity sensor, an odor sensor, a blood sensor, a urine sensor and the like, as well as any combination thereof. It is noted that a blood or urine sensor may effective sense any component of blood or urine, e.g., hemoglobin or urea and thereby effectively detect the presence of blood and/or urine.

FIG. 5 presents an example of a flow chart depicting the correspondence between the application and the sensor. According to the flow chart, the application takes a reading from the sensor, first checking whether or not biodegradable underwear (1) and/or biodegradable sanitary napkin (3) were removed. If removed, the application checks whether a new biodegradable underwear (1) and/or biodegradable sanitary napkin (3) was applied. If not, the application continues to check whether a new biodegradable underwear (1) and/or biodegradable sanitary napkin (3) was applied, until the application receives a positive answer. If a new biodegradable underwear (1) and/or biodegradable sanitary napkin (3) was applied and also if the original one was not removed (i.e., the answer to the first question in the flow chart was negative), the application checks whether biodegradable underwear (1) and/or biodegradable sanitary napkin (3) should be replaced, e.g., since it is full or close to being full or since the sensor has malfunctioned. If it is determined that replacement is not necessary, the timer is restarted for a predetermined length of time, which, when ended, the process begins again, wherein the application checks whether biodegradable underwear (1) and/or biodegradable sanitary napkin (3) were removed. If, on the other hand, the reading from the sensor is that biodegradable underwear (1) and/or the biodegradable sanitary napkin (3) should be replaced, the user is alerted by the application that biodegradable underwear (1) and/or biodegradable sanitary napkin (3) should be replaced. The user may be alerted by any appropriate means, such as an alarm, a ringtone, vibration, a message and the like. If the user reacts to the alert and/or if the sensor detects that the user reacts by replacing biodegradable underwear (1) with a new pair or by replacing biodegradable sanitary napkin (3) with a new one, the timer is restarted. If the dedicated application and sensor do not detect user reaction to the alert, the user is re-alerted until a reaction is received or until the number of alerts reaches a predefined maximal value.

According to some embodiments, the user may update the application with any appropriate data, e.g., that sensor (5) and/or biodegradable sanitary napkin (3) has been removed and/or replaced. According to some embodiments, the user may independently query the application for data regarding the condition of sensor (5), the condition of biodegradable sanitary napkin (3), the last time sensor (5) and/or biodegradable napkin (3) have been replaced, and the like.

According to some embodiments, the application may store any required or desired data, e.g., data regarding menstruation dates, the length of each menstruation period, the number of biodegradable sanitary napkins, biodegradable underwear and/or biodegradable garments used over a predefined period of time, e.g., a day, half a day, the entire menstruation period, or the like.

According to some embodiments, the sensor is disposed of together with the biodegradable underwear/biodegradable garment/biodegradable sanitary napkin and a new sensor is applied together with any of those articles. Accordingly, embodiments of the invention are directed to a designated application that automatically detects any new sensor and connects thereto. According to some embodiments, the new sensor sends a signal to the designated application, thereby allowing the designated application to detect the new sensor and to connect hereto.

According to some embodiments, the biodegradable underwear of the invention may include a belt and a biodegradable garment attached thereto. The belt may be a non-biodegradable belt or a biodegradable belt. The belt may be designed for multiple uses, may be non-disposable or disposable while the biodegradable garment attached thereto is designed to be disposable. The belt may be fastened around the body by any appropriate fastening/unfastening means, including clasps, ties, hooks, fabric hook and loop fasteners, tape, snaps, buttons, zippers and the like. According to other embodiments, the belt may be prepared from any type of material comprising enough stretch to fit around the body and be placed approximately at the waist of the user, while the belt is in circular form, and therefore, the belt itself may not need to include means to open/close/fasten/unfasten the belt itself.

The biodegradable garment may be attached to the belt by any appropriate fastening/unfastening means, including clasps, ties, hooks, fabric hook and loop fasteners, tape, snaps, buttons, zippers and the like. Further, any embodiments disclosed herein regarding the various embodiments of the biodegradable underwear may be included in the embodiments of the biodegradable garment, as appropriate.

The biodegradable garment may include a biodegradable sanitary napkin. A sensor may be included in the belt or the biodegradable garment. The sensor may be coupled to a designated application, as disclosed herein.

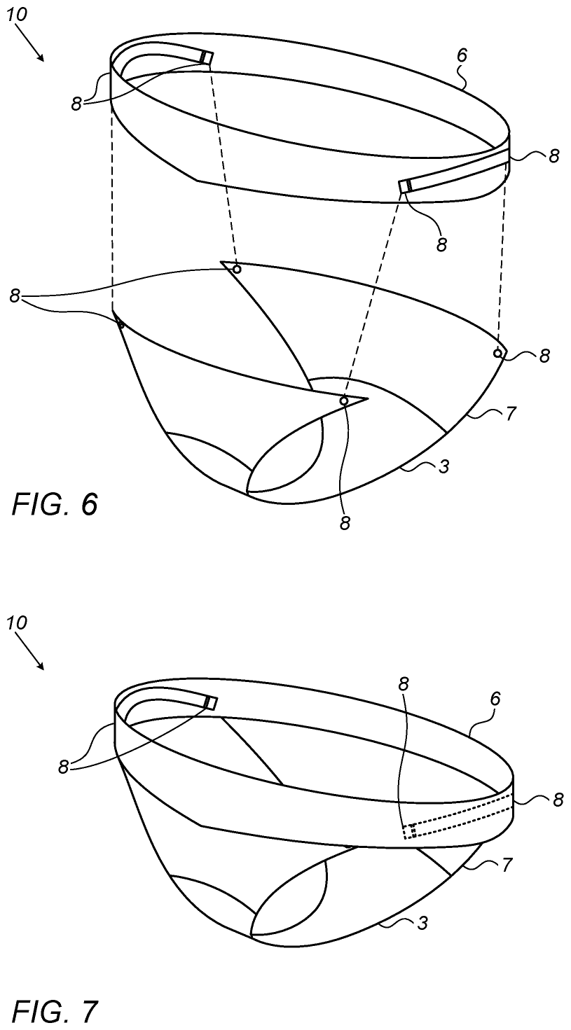

Reference is now made to FIGS. 6 and 7, presenting an embodiment of the biodegradable underwear (10) of the invention, which includes both a belt (6) and a biodegradable garment (7). FIG. 6 presents belt (6) and biodegradable garment (7) in a detached configuration, while FIG. 7 presents belt (6) and biodegradable garment (7) in the attached configuration.

Belt (6) and biodegradable garment (7) may be attached/detached from one another by fastening/unfastening means (8), which may be any appropriate means, as detailed herein. Therefore, the user may replace biodegradable garment (7) with or without replacing belt (6), which may or may not be reusable, as detailed herein.

A biodegradable sanitary napkin (3) may be incorporated into or onto biodegradable garment (7), either permanently or non-permanently, as described herein. The user may therefore replace biodegradable garment (7) together with biodegradable sanitary napkin (3).

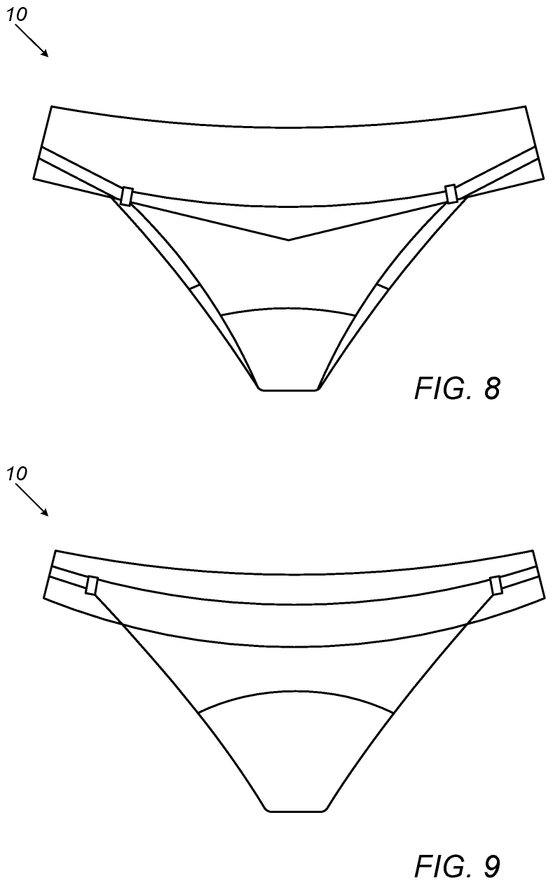

Biodegradable underwear (10) may have any appropriate shape, for comfort as well as protection. For example, as shown in FIGS. 8 (front view) and 9 (back view), the front of biodegradable underwear (10) may be narrower than the back of biodegradable underwear (10).

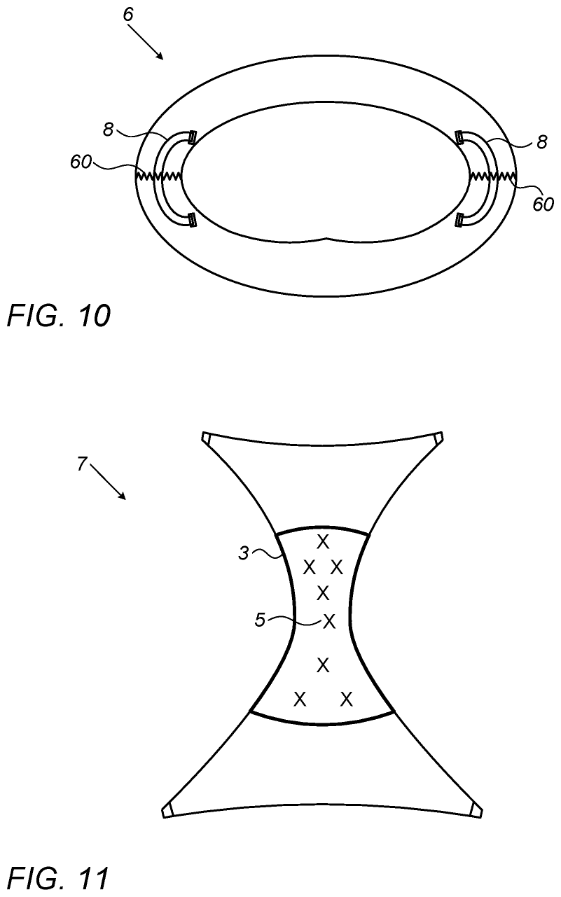

Similarly, belt (6) may have any appropriate shape, e.g., as shown in FIG. 10, presenting a top view of belt (6). According to some embodiments, belt (6) comprises fastening/unfastening means (8) for attaching/detaching the biodegradable garment (not shown) from belt (6). Belt (6) may further comprise opening/closing means (60) for opening and closing belt (6) in order to fit it onto the body. Belt (6) may include any number of opening/closing means (60), e.g., 1 or 2, possibly parallel to one another. According to some embodiments, belt (6) does not include any opening/closing means (60); rather, belt (6) may be prepared from a material having enough stretch to be appropriately positioned on the body of the user and removed therefrom without requiring opening belt (6). According to some embodiments, even if belt (6) may be positioned on the body of the user without opening belt (6), it may be removed therefrom by opening belt (6), wherein the opening/closing means (60) may be any appropriate means, as detailed herein, as well as a perforated line or any other means causing belt (6) to tear at a certain point or points when pressure is purposely exerted thereon.

According to some embodiments, sensor (5) may be incorporated into or onto sanitary napkin (3) or into or onto biodegradable garment (7). As shown in FIG. 11, presenting a top view of biodegradable garment 7, sensor (5) may be placed in the center of sanitary napkin (3) or in any other appropriate position, e.g., any of the points marked “X” in FIG. 11.

As detailed herein, sensor (5) may be any appropriate sensor, possibly connected to a designated application. The sensor may be coupled to any hardware or software by Wi-Fi, Bluetooth, or by any other appropriate means. The sensor may be optical or electrical, sensing the change in color or humidity in or on sanitary napkin (3) or biodegradable garment (7).

According to some embodiments, the sensor may include any type of conductor, including conducting ink, wherein the ink may be deployed in any appropriate pattern on sanitary napkin (3) and/or biodegradable garment (7), such that when liquid, e.g., urine or blood, comes in contact with the conducting ink, it sends a signal, e.g., to a designated application. Thus, unlike the embodiment shown in FIG. 11, sensor (5) may not be at a certain point on biodegradable garment (7) or sanitary napkin (3); rather, it may be, e.g., printed onto any region of biodegradable garment (7), sanitary napkin (3), or both, in any desired pattern. Such embodiments of sensor (5) may be used with biodegradable underwear (1) as well.

The inventions illustratively described herein may suitably be practiced in the absence of any element or elements, limitation or limitations, not specifically disclosed herein. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise indicated.

1-27. (canceled)

28. A biodegradable sanitary napkin, comprising:
a biodegradable material configured to absorb menstrual blood; and
at least one sensor for detecting the presence of blood, wherein the at least one sensor is incorporated into the biodegradable sanitary napkin,
and wherein the at least one sensor is configured to transmit data indicative of a detection of the presence of blood in the biodegradable sanitary napkin.

29. The biodegradable sanitary napkin of claim 28, wherein the sensor is configured to sense at least one blood component.

30. The biodegradable sanitary napkin of claim 28, wherein the at least one sensor is in communication with an application running on a user mobile device.

31. The biodegradable sanitary napkin of claim 30, wherein the application is configured to receive the data from the sensor and alert a user that blood was detected in the biodegradable sanitary napkin.

32. The biodegradable sanitary napkin of claim 28, wherein the sanitary napkin is prepared from one of: seaweed, zinc, a biodegradable polymer, and any combination thereof

33. The biodegradable sanitary napkin of claim 28, further comprising one or more attachment means for attaching the sanitary napkin to a garment.

34. A system comprising:
a sanitary napkin comprising biodegradable material configured to absorb menstrual blood;
one or more attachment means for attaching the base sanitary napkin to a garment;
at least one sensor for detecting the presence of blood, incorporated into the biodegradable sanitary napkin; and
an application running on a mobile user device and configured to receive data from the sensor and to alert a user that blood was detected in the biodegradable sanitary napkin.

35. The system of claim 34, wherein the sensor is configured to sense at least one blood component.

36. The system of claims 34, wherein the sanitary napkin is prepared from one of:
seaweed, zinc, a biodegradable polymer and any combination thereof

37. A biodegradable underwear, comprising:
a biodegradable garment;
a biodegradable sanitary napkin incorporated into or onto the biodegradable garment,
wherein the sanitary napkin comprises biodegradable material configured to absorb menstrual blood; and
at least one sensor for detecting the presence of blood, wherein the at least one sensor is incorporated into the biodegradable sanitary napkin,
and wherein the at least one sensor is configured to transmit data indicative of a detection of the presence of blood in the biodegradable sanitary napkin.

38. The biodegradable underwear of claim 37, wherein the sensor is configured to sense at least one blood component.

39. The biodegradable underwear of claim 37, wherein the sensor is in communication with an application running on a user mobile device.

40. The biodegradable underwear of claim 39, wherein the application is configured to receive data from the sensor and alert a user that blood was detected in the biodegradable sanitary napkin.

41. The biodegradable underwear of claim 37, wherein the sanitary napkin is prepared from one of: seaweed, zinc, a biodegradable polymer, and any combination thereof

42. The biodegradable underwear of claim 37, wherein the garment is prepared from one of: seaweed, zinc, a biodegradable polymer, and any combination thereof
